(19) United States
(12) Patent Application Publication
(10) Pub. No.: US 2025/0069755 A1
(43) Pub. Date: Feb. 27, 2025

(54) METHOD OF ESTIMATING HEALTH SCORE AND METHOD OF ESTIMATING CHANGES IN POSITIONS OF PARTS OF UPPER BODY

(71) Applicants: TOYODA GOSEI CO., LTD., Kiyosu-shi (JP); NATIONAL UNIVERSITY CORPORATION TOKAI NATIONAL HIGHER EDUCATION AND RESEARCH SYSTEM, Nagoya-shi (JP)

(72) Inventors: Takeshi FUJIWARA, Kiyosu-shi (JP); Hitoshi Hirata, Nagoya-shi (JP); Shingo Shimoda, Nagoya-shi (JP)

(21) Appl. No.: 18/680,720

(22) Filed: May 31, 2024

(30) Foreign Application Priority Data
Jun. 27, 2023 (JP) 2023-105150
Dec. 26, 2023 (JP) 2023-219010

(51) Int. Cl. G16H 50/30; A61B 5/00; A61B 5/11; G16H 10/20; G16H 10/60; G16H 50/70

(52) U.S. Cl. CPC G16H 50/30; A61B 5/1116; A61B 5/112; A61B 5/1126; A61B 5/4528; A61B 5/4842; A61B 5/6807; A61B 5/743; G16H 10/20; G16H 10/60; G16H 50/70; A61B 2562/0247; A61B 2562/046

(57) ABSTRACT

A method of estimating a health score is disclosed. The health score includes a knee condition score for quantitatively evaluating a knee condition of a user. The estimating method includes acquiring a data provider feature that is a feature correlated with the knee condition from measurement information indicating temporal changes in forces applied to multiple parts of a sole of each of multiple data providers, generating a learning model by learning a correlation between the data provider feature and the knee condition, acquiring a user feature from measurement information indicating temporal changes in forces applied to multiple parts of a sole of the user, and estimating the knee condition of the user as the knee condition score by providing the learning model with the user feature.

### CROSS-REFERENCE TO RELATED APPLICATIONS

This application is based upon and claims the benefit of priorities from prior Japanese Patent Application No. 2023-105150, filed on Jun. 27, 2023, and prior Japanese Patent Application No. 2023-219010, filed on Dec. 26, 2023, the entire contents of each of which are incorporated herein by reference.

### BACKGROUND

### 1. Field

The present disclosure relates to a method of estimating a health score for quantitatively evaluating a health status related to walking of a user. The present disclosure also relates to a method of estimating how positions of multiple parts of the upper body of a user change over time during walking.

### 2. Description of Related Art

In recent years, conditions that require nursing care due to locomotor disability and locomotive syndrome, which is a state with a high risk of requiring nursing care, have become a social issue. In order to detect locomotive syndrome at an early stage, techniques for determining locomotive syndrome have been developed. For example, Japanese Laid-Open Patent Publication No. 2021-137371 discloses a system capable of readily recognizing changes in the gait condition of a user.

This system measures pressures applied to multiple parts of the sole over time. The system acquires features related to the gait condition from the acquired measurement information. Based on the acquired features, the system determines whether the gait condition is in a specified condition targeted for alert. The system is configured to issue a notification when it determines that the gait condition is the specified condition targeted for alert.

According to the above-described system, the user walks while wearing a measurement device. Alternatively, another person, such as a guardian, causes the user to wear the measurement device and walk. The user or another person then checks whether there is a notification indicating that the gait condition of the user is in the condition targeted for alert. The notification allows the user or another person to readily recognize that the gait condition of the user has changed to the condition targeted for alert without the need for direct diagnosis by a health professional. Consequently, the user can take appropriate actions such as visiting a medical facility. Additionally, the other person can also take appropriate actions for the user, such as taking them to a medical facility.

Using the features related to the gait condition, it is desirable to estimate a score for quantitatively evaluating the health status related to each user's gait, such as the knee condition. This would allow for early detection of knee deterioration, and consequently, early signs of disease. However, a method of estimating such a score has not yet been realized.

Moreover, the technology disclosed in the above-described is configured to exclusively diagnoses changes in the condition of the lower limbs as the gait condition. However, in recent years, there is a need to estimate not only changes in the state of the lower limbs but also changes in the state of the upper body, especially changes in the positions of multiple parts over time.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of estimating a health score is provided. The health score is used to quantitatively evaluate a health status related to walking of a user. The health score includes a knee condition score for quantitatively evaluating a knee condition of the user. The estimating method includes: acquiring a data provider feature that is a feature related to a gait condition of each of multiple data providers and is a feature correlated with the knee condition from measurement information indicating temporal changes in forces applied to multiple parts of a sole of each of the multiple data providers; generating a learning model by learning a correlation between the acquired data provider feature and the knee condition from the data provider feature and data on the knee condition of each data provider; acquiring a user feature that is a feature of the same type as the data provider feature from measurement information indicating temporal changes in forces applied to multiple parts of a sole of the user; and estimating the knee condition of the user as the knee condition score by providing the learning model with the acquired user feature.

In another general aspect, a method of estimating a health score is provided. The health score is used to quantitatively evaluate a health status related to walking of a user. The health score includes a gait age score for quantitatively evaluating a gait age of the user. The estimating method includes: acquiring a data provider feature that is a feature related to a gait condition of each of multiple data providers and is a feature correlated with the gait age from measurement information indicating temporal changes in forces applied to multiple parts of a sole of each of the multiple data providers; generating a learning model by learning a correlation between the acquired data provider feature and the gait age from the data provider feature and data on the gait age of each data provider; acquiring a user feature that is a feature of the same type as the data provider feature from measurement information indicating temporal changes in forces to multiple parts of a sole of the user; and estimating the gait age of the user as the gait age score by providing the learning model with the acquired user feature.

In yet another general aspect, a method of estimating changes in positions of parts of upper body is provided. The method includes: measuring, when each of multiple data providers walks, temporal changes in forces applied to multiple parts of a sole of the data provider by using a measurement device; measuring, in synchronization with the measurement of the temporal changes in the forces, temporal changes in positions of multiple parts of the upper body of the data provider; generating a learning model by learning a correlation between the measured temporal changes in the forces and the measured temporal changes in the positions of the multiple parts; measuring, when a user walks, temporal changes in forces applied to multiple parts of a sole of the user by using the same measurement device used for measurement for the data provider; and estimating temporal changes in positions of multiple parts of the upper body of the user by providing the learning model with the temporal changes in the forces measured for the user.

Other features and aspects will be apparent from the following detailed description, the drawings, and the claims.

### BRIEF DESCRIPTION OF THE DRAWINGS

FIG. 1 is a two-dimensional map defining a relationship of a health status to a knee condition score and a gait age score in a first embodiment.

FIG. 2 is an explanatory diagram of a gait determination system in the first embodiment.

FIG. 3 is an explanatory diagram of an arrangement of pressure sensors in a running determination system shown in FIG. 2.

FIG. 4 is an explanatory diagram of a feature data set based on measurement information acquired from the pressure sensors shown in FIG. 3.

FIG. 5 is an explanatory diagram of an information processing unit in the running determination system shown in FIG. 2.

FIG. 6 is an explanatory diagram of a score estimation unit in the running determination system shown in FIG. 2.

FIG. 7 is a graph of waveform data showing temporal changes in pressures measured by the pressure sensors shown in FIG. 3.

FIG. 8 is a graph of waveform data of a one-step interval extracted from the graph of FIG. 7.

FIG. 9 is a graph of the two-dimensional coordinates of the center of foot pressure.

FIG. 10 is an explanatory view of a swing angle.

FIG. 11 is an explanatory flowchart of a procedure for estimating a knee condition score and a gait age score.

FIG. 12 is an explanatory diagram of a data provider measurement step in a second embodiment.

FIG. 13 is an explanatory diagram of an arrangement state of pressure sensors in insoles of the second embodiment.

FIG. 14 is a graph showing an example of pressure waveforms measured by the pressure sensors of the second embodiment.

FIG. 15 is an explanatory diagram of a user measurement step in the second embodiment.

FIG. 16 is an explanatory flowchart of a procedure for estimating temporal changes in the positions of multiple parts of the upper body of the user in the second embodiment.

FIG. 17 is a diagram showing a display that illustrates markers respectively corresponding to multiple parts of the user in the second embodiment.

FIG. 18 is a diagram showing a display that illustrates a trajectory of displacement of a marker corresponding to a specific part in the lumbar region of the user in the second embodiment.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

This description provides a comprehensive understanding of the methods, apparatuses, and/or systems described. Modifications and equivalents of the methods, apparatuses, and/or systems described are apparent to one of ordinary skill in the art. Sequences of operations are exemplary, and may be changed as apparent to one of ordinary skill in the art, with the exception of operations necessarily occurring in a certain order. Descriptions of functions and constructions that are well known to one of ordinary skill in the art may be omitted.

Exemplary embodiments may have different forms, and are not limited to the examples described. However, the examples described are thorough and complete, and convey the full scope of the disclosure to one of ordinary skill in the art.

In this specification, “at least one of A and B” should be understood to mean “only A, only B, or both A and B.”

A first embodiment of the present disclosure will now be described with reference to the drawings. The first embodiment relates to a method of estimating a health score.

The estimating method of the present embodiment is implemented in a gait determination system.

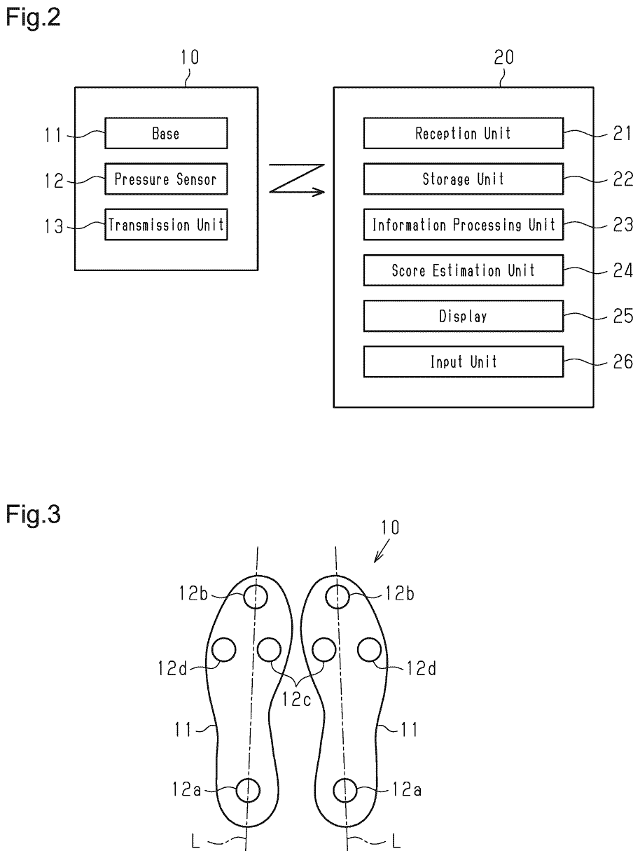

As shown in FIG. 2, the gait determination system includes a measurement device 10 and a gait determination device 20. Next, each component of the gait determination system will be described.

### Measurement Device 10

As shown in FIGS. 2 and 3, the measurement device 10 includes a base 11 used as an insole of a shoe. Multiple pressure sensors 12 for detecting pressures applied to multiple parts of the sole during walking are attached to the base 11. The pressure sensors 12 include, for example, a heel-side sensor 12a, a toe-side sensor 12b, an inner-side sensor 12c, and an outer-side sensor 12d.

Walking generally includes two phases: the stance phase, during which the soles touch the ground to support the body, and the swing phase, during which each foot is lifted and swung forward. In this specification, in addition to the stance phase and the swing phase, a period of standing still in a static upright position immediately before starting to move and a period immediately after movement has ended are also considered part of walking.

As shown in FIG. 3, the heel-side sensor 12a is disposed on a portion of the base 11 to which the load of the heel is applied, and detects the pressure applied to the heel of the sole of the foot. In order to distinguish the five toes, the toes are referred to as a first toe, a second toe, a third toe, a fourth toe, and a fifth toe in the order from the big toe, which is the first toe, to the little toe, which is the fifth toe. The toe-side sensor 12b is disposed in any portion of the region of the second to fourth toes where a load is applied, and detects the pressure applied to the toes of the sole.

The inner-side sensor 12c is disposed on the inner side of a line L connecting the heel-side sensor 12a and the toe-side sensor 12b and at a portion of the base 11 to which the load of the ball of the big toe is applied. The inner-side sensor 12c detects the pressure applied to the inner part of the sole. The outer-side sensor 12d is disposed on the outer side of the line L and at a portion of the base 11 to which the load of the ball of the little toe is applied. The outer-side sensor 12d detects the pressure applied to the outer part of the sole. The inner side of each foot refers to a side closer to the other foot. The outer side of each foot refers to a side farther from the other foot.

The heel-side sensor 12a and the toe-side sensor 12b are respectively disposed to detect the pressure at a first position and the pressure at a second position, which are spaced apart from each other in the longitudinal direction on the sole. The inner-side sensor 12c and the outer-side sensor 12d are disposed so as to respectively detect the pressure at a third position and the pressure at a fourth position, which are separated from each other in the lateral direction with the line L connecting the first position and the second position therebetween.

The heel-side sensor 12a, the toe-side sensor 12b, the inner-side sensor 12c, and the outer-side sensor 12d may simply be referred to as pressure sensors 12 when they do not need to be distinguished from each other.

Each of the pressure sensors 12 independently detects the pressure applied to a corresponding part of the sole at specified time intervals. The specified time is, for example, 5 milliseconds to 30 milliseconds.

The pressure sensors 12 may be, for example, known pressure-sensitive sensors using piezoelectric elements or the like. Particularly, considering the use situation, where the sensors are placed on the soles, it is preferable to use capacitive sensors made of elastomer that utilizes dielectric elastomer, from the viewpoints of flexibility and durability. The dielectric elastomer may be, for example, a crosslinked polyrotaxane, a silicone elastomer, an acrylic elastomer, or a urethane elastomer.

Capacitive sensors made of elastomer can be formed thinly, which offers the advantage that such sensors do not significantly increase the thickness of base 11, which is used as an insole, when the sensors are incorporated in the insole. This type of sensor has a structure in which dielectric elastomer is placed between a pair of electrodes. When the dielectric elastomer is deformed by tension or stress, the capacitance, which is the electric charge stored in the electrodes, changes. This change in capacitance is measured as the pressure applied to the sole.

As shown in FIG. 2, a transmission unit 13 is attached to the measurement device 10. The transmission unit 13 transmits a detected value acquired by each pressure sensor 12 to the gait determination device 20 as measurement information at specified times, for example, at each detection timing. The detected value acquired by each pressure sensor 12 can be converted into a pressure value corresponding to a detection method of the pressure sensor, such as a capacitance value or an electric resistance value. Therefore, the term “detected values” mentioned below can be referred to as the measured values of pressure at the locations of the sole where the pressure sensors 12 are disposed.

The measurement device 10 is prepared for each of the left foot and the right foot. If necessary, only one of the left and right measurement devices 10 or both of the left and right measurement devices 10 are used.

### Gait Determination Device 20

As shown in FIG. 2, the gait determination device 20 includes a reception unit 21, which receives the measurement information transmitted from the transmission unit 13 of the measurement device 10, a storage unit 22, which stores various kinds of data such as the received measurement information, and an information processing unit 23, which acquires features related to a gait condition from the measurement information. In addition, the gait determination device 20 includes a score estimation unit 24, which estimates a health score for quantitatively evaluating the health status related to the gait of a user based on the acquired features. In the present embodiment, the health status includes the knee condition of the user and the gait age of the user. That is, in the present embodiment, the health score includes a knee condition score for quantitatively evaluating the knee condition of the user and a gait age score for quantitatively evaluating the gait age of the user.

The gait determination device 20 additionally includes a display 25 for showing various types of information, and an input unit 26 for entering various types of information.

The gait determination device 20 may be a computer, such as a mobile terminal or a tablet terminal. The display 25 may be a known display device, such as an LCD display. The input unit 26 may be a known input device, such as a keyboard, a mouse, or a touch screen.

The reception unit 21 includes a wired or wireless communication device and communicates with the transmission unit 13 of the measurement device 10 by a known communication method.

### Storage Unit 22

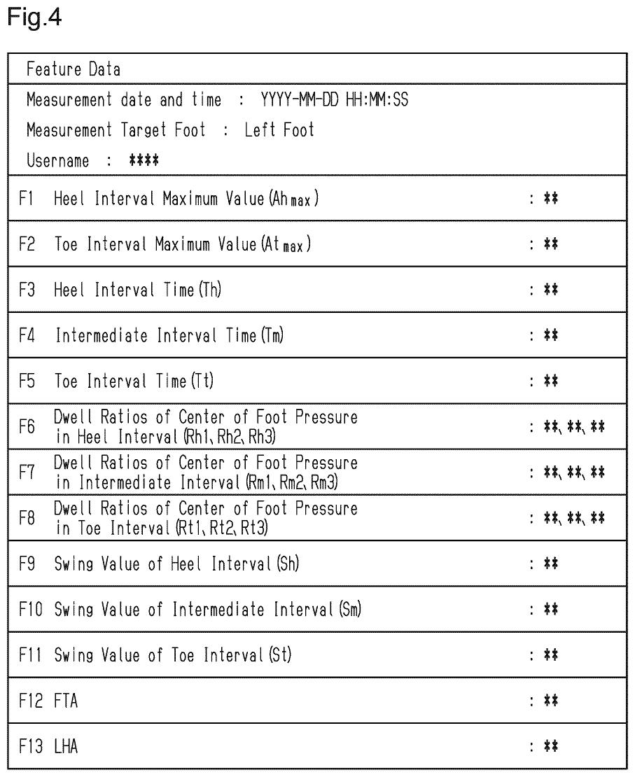

The storage unit 22 is configured to store the measurement information transmitted from the measurement device 10 and to store the feature data set based on the measurement information. As illustrated in FIG. 4, the feature data set includes identification information such as date and time and a user name, and features F1 to F13 related to the gait condition.

The storage unit 22 stores multiple feature data sets such as past feature data sets of the same data provider, feature data sets of multiple different data providers, and a model data set provided by an expert such as a health professional. The model data set may be, for example, a feature data set acquired from measurement results of a data provider having a specific symptom such as talipes varus or talipes valgus. Further, the storage unit 22 stores programs for causing the information processing unit 23 and the score estimation unit 24 to execute various processes.

The storage unit 22 may be, for example, an HDD, an SSD, or a semiconductor memory device. The storage unit 22 may be connected to the gait determination device 20 via a network.

### Information Processing Unit 23

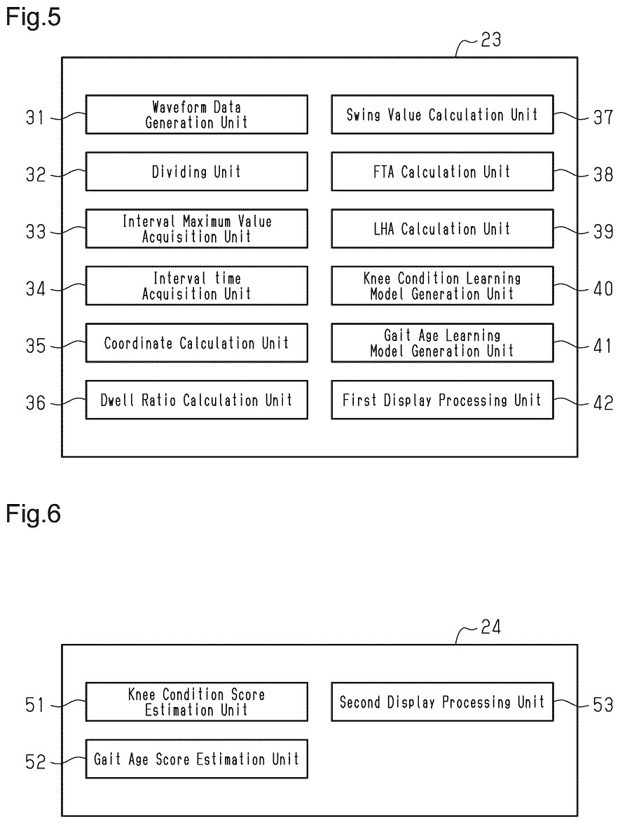

As illustrated in FIG. 5, the information processing unit 23 includes a waveform data generation unit 31, a dividing unit 32, an interval maximum value acquisition unit 33, an interval time acquisition unit 34, a coordinate calculation unit 35, a dwell ratio calculation unit 36, a swing value calculation unit 37, an FTA calculation unit 38, and an LHA calculation unit 39. Additionally, the information processing unit 23 includes a knee condition learning model generation unit 40, a gait age learning model generation unit 41, and a first display processing unit 42. The information processing unit 23 may be, for example, processing circuitry such as a computer including a CPU.

### Waveform Data Generation Unit 31

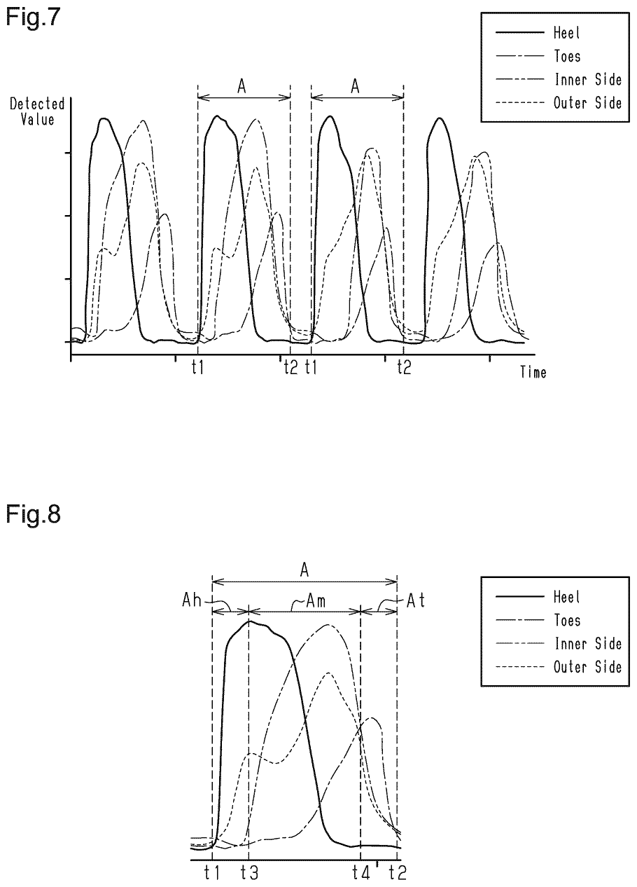

The waveform data generation unit 31 generates waveform data representing temporal changes in detected values acquired by the respective pressure sensors 12 corresponding to one foot based on the measurement information to be analyzed, which is stored in the storage unit 22. FIG. 7 shows an example of the waveform data generated by the waveform data generation unit 31. In FIG. 7, the solid line indicates a detected value of the heel-side sensor 12a, the long-dash short-dash line indicates a detected value of the toe-side sensor 12b, the long-dash double-short-dash line indicates a detected value of the inner-side sensor 12c, and the broken line indicates a detected value of the outer-side sensor 12d.

### Dividing Unit 32

The dividing unit 32 extracts a specific step interval, from footstrike to toe-off during walking, from the generated waveform data and further divides this extracted target interval into multiple smaller subintervals.

As shown in FIG. 7, the waveform data acquired during walking periodically repeats an interval in which the detected value is substantially constant and an interval in which the detected value changes. In the waveform data during walking, an interval in which the detected value is substantially constant is an interval in which the foot is separated from the ground, that is, corresponds to a swing phase. In the waveform data, an interval in which the detected value changes is an interval in which the foot is in contact with the ground. The dividing unit 32 extracts a specific one-step interval A from multiple one-step intervals A. A time point at which the detected values corresponding to the heel start to rise from a state in which the detected values corresponding to multiple parts are substantially constant is defined as a start point t1 of the one-step interval A. The latest time point among the respective time points at which the detected values corresponding to the multiple parts start to be substantially constant is defined as an end point t2 of the one-step interval A.

The start point t1 and the end point t2 of the one-step interval A may be defined by a method different from that described above. For example, the end point t2 may be set to a time point at which a specified time has elapsed after the detected values corresponding to multiple parts become substantially constant.

The dividing unit 32 selects the one-step interval A to be extracted, for example, based on a criterion set in advance as described below. The dividing unit 32 selects the one-step interval A located in a preset order, such as the tenth step, the twentieth step, and so on, from the start of measurement. Alternatively, the dividing unit 32 selects the one step interval A that comes after the elapse of a certain time from the previously extracted one step interval A. Alternatively, the dividing unit 32 divides the waveform data by a certain number of steps or time, and randomly selects a one-step interval A in each divided interval.

As illustrated in FIG. 8, the dividing unit 32 divides the extracted one-step interval A into multiple subintervals such as a heel interval Ah, a toe interval At, and an intermediate interval Am.

The heel interval Ah is an initial subinterval of the one-step interval A in which a relatively large pressure is applied to the heel side. In the present embodiment, the heel interval Ah is set to an interval from the start point t1 of the one-step interval A to a time point t3, at which the detected value of the heel-side sensor 12a reaches a peak.

The toe interval At is a final subinterval of the one-step interval A in which a relatively large pressure is applied to the toe side. In the present embodiment, the toe interval At is set to an interval from a time point t4, at which the detected value of the toe-side sensor 12b becomes the largest among the detected values of the four pressure sensors 12, to the end point t2 of the one-step interval A. Depending on the way of walking, the toe interval At may not be provided.

The intermediate interval Am is a subinterval acquired by removing the heel interval Ah and the toe interval At from the one-step interval A. When the toe interval At exists, the interval from the time point t3 to the time point t4 is defined as the intermediate interval Am. When the toe interval At does not exist, the interval from the time point t3 to the end point t2 is defined as the intermediate interval Am.

The heel interval Ah, the intermediate interval Am, and the toe interval At may be defined differently from the above. For example, the toe interval At may be set to an interval from a time point at which the detected value of the toe-side sensor 12b reaches a peak to the end point t2 of the one-step interval A.

Each subinterval may be defined without using the relationship between the pressures on multiple parts. For example, the heel interval Ah may be set to an interval from the start point t1 of the one-step interval A to a point at which a specific time has elapsed from the start point t1. The toe interval At may be set to an interval from a specific time before the end point t2 of the one-step interval A to the end point t2.

Each one-step interval A may be divided into multiple subintervals by a method different from that described above. For example, the one-step interval A may be divided into four or more subintervals by further dividing the intermediate interval Am. The multiple subintervals may be subintervals divided independently of an interval in which a relatively large pressure is applied to the heel side and an interval in which a relatively large pressure is applied to the toe side.

### Interval Maximum Value Acquisition Unit 33

The interval maximum value acquisition unit 33 acquires, from the measurement information, the maximum value (hereinafter, referred to as a heel interval maximum value Ahmax) among the detected values of the four pressure sensors 12 in the heel interval Ah and the maximum value (hereinafter, referred to as a toe interval maximum value Atmax) among the detected values of the four pressure sensors 12 in the toe interval At. Then, the interval maximum value acquisition unit 33 stores the acquired heel interval maximum value Ahmax and toe interval maximum value Atmax in the storage unit 22 as the features F1 and F2 in the feature data set shown in FIG. 4.

### Interval Time Acquisition Unit 34

The interval time acquisition unit 34 acquires, from the measurement information, an interval time Th, which is the time length of the heel interval Ah, an interval time Tm, which is the time length of the intermediate interval Am, and an interval time Tt, which is the time length of the toe interval At. Then, the interval time acquisition unit 34 stores the acquired interval times Th, Tm, and Tt in the storage unit 22 as the features F3 to F5 in the feature data set illustrated in FIG. 4. Each interval time may be a time itself such as the number of seconds or may be a parameter corresponding to the interval time such as the number of measurement points in the corresponding interval.

### Coordinate Calculation Unit 35

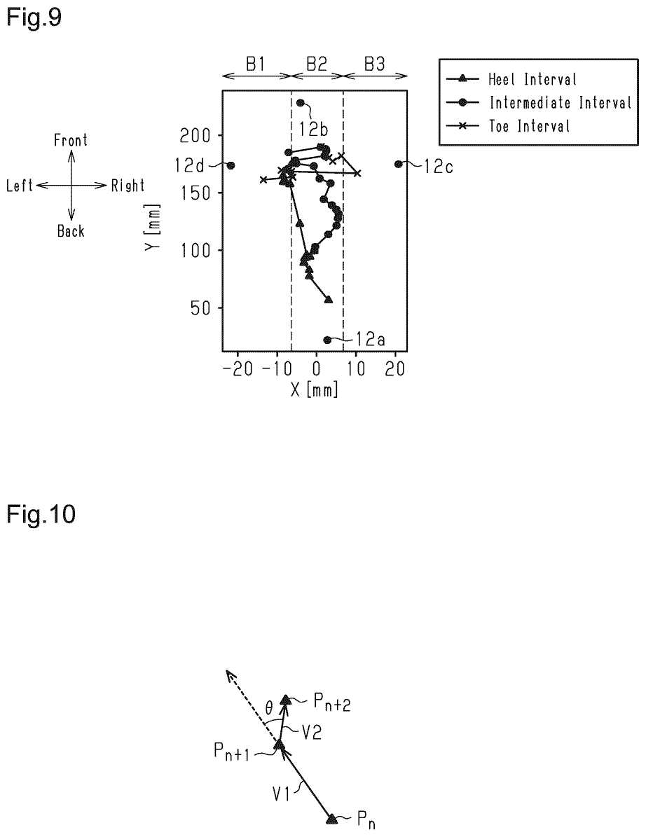

As shown in FIG. 9, the coordinate calculation unit 35 calculates the two-dimensional coordinates (X (t), Y (t)) of the center of foot pressure acquired for each detection time in each of the heel interval Ah, the toe interval At, and the intermediate interval Am based on the measurement information of the one-step interval A. X(t) represents the lateral coordinate of the center of foot pressure at time t, and Y(t) represents the longitudinal coordinate of the center of foot pressure at time t. The two-dimensional coordinates of the center of foot pressure can be acquired from the detected values of the four pressure sensors 12 detected at the same detection time. Points 12a to 12d in the two-dimensional coordinate system illustrated in FIG. 9 represent the positions of the pressure sensors 12.

### Dwell Ratio Calculation Unit 36

As shown in FIG. 9, the dwell ratio calculation unit 36 divides the two-dimensional coordinate system of the center of foot pressure into three preset regions B1, B2, and B3. The dwell ratio calculation unit 36 calculates a dwell ratio Rh1, a dwell ratio Rh2, and a dwell ratio Rh3, based on the two-dimensional coordinates calculated by the coordinate calculation unit 35. The dwell ratio Rh1 is a ratio of time during which the two-dimensional coordinates of the center of foot pressure remain within the region B1 in the heel interval Ah. The dwell ratio Rh2 is a ratio of time during which the two-dimensional coordinates of the center of foot pressure remain within the region B2 in the heel interval Ah. The dwell ratio Rh3 is a ratio of time during which the two-dimensional coordinates of the center of foot pressure remain within the region B3 in the heel interval Ah. The calculated dwell ratios Rh1, Rh2, and Rh3 are stored in the storage unit 22 as the feature F6 in the feature data set shown in FIG. 4.

Similarly, the dwell ratio calculation unit 36 calculates, in the intermediate interval Am, a dwell ratio Rm1, which is a ratio of time during which the two-dimensional coordinates of the center of foot pressure remain within the region B1, a dwell ratio Rm2, which is a ratio of time during which the two-dimensional coordinates of the center of foot pressure remain in the region B2, and a dwell ratio Rm3, which is a ratio of time during which the two-dimensional coordinates of the center of foot pressure remain in the region B3. The calculated dwell ratios Rm1, Rm2, and Rm3 are stored in the storage unit 22 as the feature F7 in the feature data set shown in FIG. 4.

Similarly, the dwell ratio calculation unit 36 calculates, in the toe interval At, a dwell ratio Rt1, which is a ratio of time during which the two-dimensional coordinates of the center of foot pressure remain within the region B1, a dwell ratio Rt2, which is a ratio of time during which the two-dimensional coordinates of the center of foot pressure remain in the region B2, and a dwell ratio Rt3, which is a ratio of time during which the two-dimensional coordinates of the center of foot pressure remain in the region B3. The calculated dwell ratios Rt1, Rt2, and Rt3 are stored in the storage unit 22 as the feature F8 in the feature data set shown in FIG. 4.

Each of the dwell ratios may be calculated either based on the actual duration, such as the number of seconds the two-dimensional coordinates of the center of foot pressure remain within the corresponding region, or based on parameters equivalent to dwell time, such as the number of measurement points located within the region in the two-dimensional coordinate system.

When the dwell ratios are calculated, the two-dimensional coordinate system of the center of foot pressure may be divided by a method different from the above-described method. The two-dimensional coordinate system may be divided into multiple regions arranged in the longitudinal direction, or may be divided into multiple regions arranged in the longitudinal and lateral directions, instead of being divided into multiple regions arranged in the lateral direction. The number of divisions of the two-dimensional coordinate system of the center of foot pressure may be two, or may be greater than three.

### Swing Value Calculation Unit 37

The swing value calculation unit 37 calculates a swing value Sh of the heel interval Ah, a swing value Sm of the intermediate interval Am, and a swing value St of the toe interval At as swing values indicating swing of the center of foot pressure based on the two-dimensional coordinates calculated by the coordinate calculation unit 35. For example, the swing value Sh of the heel interval Ah is calculated as follows.

In FIG. 10, points Pn, Pn+1, and Pn+2 are points expressed by two-dimensional coordinates of the center of foot pressure at three consecutive measurement points in the heel interval Ah. An angle formed by a vector V1, which indicates the amount of change from the point Pn to the point Pn+1 and a vector V2, which indicates the amount of change from the point Pn+1 to the point Pn+2, is defined as a swing angle θ.

The swing value calculation unit 37 define, as a point Pn, each of all the points of the two-dimensional coordinates of the center of foot pressures in the heel interval Ah, excluding the last two sets of coordinates, and calculates the swing angle θ corresponding to each point Pn. The swing value calculation unit 37 calculates the swing value Sh, which is the sum of the swing angles θ, by summing all of the calculated swing angles θ. The swing value calculation unit 37 calculates the swing value Sm of the intermediate interval Am and the swing value St of the toe interval At in the same manner. The swing value calculation unit 37 stores the calculated swing values Sh, Sm, and St in the storage unit 22 as the features F9 to F11 in the feature data set shown in FIG. 4.

Instead of calculating the swing values of all the subintervals, only the swing value of a specific subinterval may be calculated.

### FTA Calculation Unit 38

The FTA calculation unit 38 calculates a femorotibial angle (hereinafter, referred to as FTA) of a data provider or a user based on the measurement information of the one-step interval A. The FTA calculation unit 38 stores the calculated FTA in the storage unit 22 as the feature F12 in the feature data set shown in FIG. 4.

The FTA is an angle formed by the long axis of the femur and the long axis of the tibia, and is used as one of criteria for determining talipes varus and talipes valgus. Since the FTA is a parameter indicating the knee joint condition, the FTA is closely related to the gait condition. The FTA can be estimated from the pressure distribution of the sole, in particular, the balance of loads detected by the heel-side sensor 12a, the toe-side sensor 12b, the inner-side sensor 12c, and the outer-side sensor 12d.

### LHA Calculation Unit 39

The LHA calculation unit 39 calculates the leg heel angle (hereinafter, referred to as LHA) of a data provider or a user based on the measurement information of the one-step interval A. The LHA calculation unit 39 stores the calculated LHA in the storage unit 22 as the feature F13 in the feature data set shown in FIG. 4.

The LHA is an angle formed by the long axis of the lower leg and the axis of the calcaneus, and is used as one of criteria for determining talipes varus and talipes valgus. Since the LHA is a parameter indicating the inclination of the foot and the heel, the LHA is closely related to the gait condition. The LHA can be estimated from the pressure distribution of the sole, in particular, the balance of loads detected by the heel-side sensor 12a, the toe-side sensor 12b, the inner-side sensor 12c, and the outer-side sensor 12d.

One example of a method for estimating the LHA and the FTA is a method using an angle estimation model. In this method, first, training data including a set of actual measurement results of the LHA and the FTA, basic data, and gait data is collected. The basic data includes biological information such as sex, age, height, and weight. The gait data includes gait information measured by the pressure sensors 12, such as lateral balance, longitudinal balance, accelerations of footstrike and toe-off, intensities (load values) of footstrike and toe-off, and swing amplitude. Next, the collected training data is subjected to machine learning by a neural network to construct an angle estimation model for estimating and outputting the measurement results of the LHA and the FTA for the input basic data and gait data. Using such a trained angle estimation model allows for the estimation of the LHA and the FTA based on the measurement information of the data provider or the user. The swing amplitude, which is the magnitude of the swing of the body, is greatly related to the LHA and FTA, which are affected by changes in the conditions of locomotors. Therefore, incorporating swing amplitude into the gait information for training enhances the estimation accuracy of the angle estimation model.

Features other than those described above may be calculated and stored in the storage unit 22. The corresponding features are, for example, the walking speed, the synchronization of the right and left feet during walking, the continuous walking time, or the like.

### First Display Processing Unit 42

The first display processing unit 42 generates an image or the like for displaying the feature data set shown in FIG. 4, the waveform data of the one-step interval A shown in FIG. 8, the two-dimensional coordinate data of the center of foot pressure shown in FIG. 9, and the like, on the display 25. When a specified operation is performed through the input unit 26, the first display processing unit 42 causes the display 25 to display the generated image or the like.

### Knee Condition Learning Model Generation Unit 40

The knee condition learning model generation unit 40 generates a learning model by learning the correlation between the features and the knee condition from the features acquired for each data provider and the data of the knee condition of the data provider. When the learning model is generated, a feature having a positive correlation with the knee condition is selected from the above-described many kinds of features and used for the learning model. The acquisition of the feature here includes storing the feature in the storage unit 22 and reading the feature from the storage unit 22, in addition to the acquisition itself.

The data on the knee condition of the data providers includes results of responses of the data providers to a questionnaire regarding the knee condition. As the questionnaire, for example, the Japan Knee Osteoarthritis Measure (JKOM) can be used. The JKOM is a self-administered questionnaire consisting of twenty-five questions in four categories: “knee pain and stiffness”, “daily life condition”, “usual activities”, and “health condition”. The total score (25 to 125 points) is considered as the JKOM score, with higher scores indicating more severe conditions. In addition to the above questionnaire, a questionnaire called Locomo 25, which consists of twenty-five questions about problems of locomotors. Locomo is an abbreviation for locomotive syndrome, which refers to a condition where mobility (standing, walking) is impaired due to locomotor disabilities.

Any one of the JKOM and the Locomo 25 may be used, or both of them may be used in combination. In the latter case, for example, a weighted average value of the response results may be used as the data on the knee condition used in the learning model.

Further, the data on the knee condition of the data providers includes information on a classification (stage classification) indicating the degree of progress of a knee disease, for example, knee osteoarthritis. As this classification, for example, Kellgren-Lawrence classification can be used. Knee osteoarthritis is diagnosed through X-ray examination. The knee condition is assessed by focusing on the ends of the femur and tibia as shown in the X-ray images. The knee condition may include the presence and extent of the gap between the femur and tibia, the presence and degree of bowlegs or knock-knees, and the presence and severity of osteophytes (abnormally protruding bones). In the Kellgren-Lawrence classification, the condition of the knee is graded from 0 to 4 by medical professionals, to determine the progression of the knee disease (knee osteoarthritis).

Any one of the information on the response results of the questionnaire (JKOM, Locomo 25) and the information on the Kellgren-Lawrence classification may be used, or both of them may be used in combination. In the latter case, for example, the multiplication of the two sets of information may be calculated. As the number of types to be combined increases, the accuracy of the data on the knee condition, and thus the accuracy of the learning model to be generated, increases.

### Gait Age Learning Model Generation Unit 41

The gait age learning model generation unit 41 generates a learning model by learning the correlation between the features and the gait age from the features acquired for each data provider and the data of the gait age of the data providers. A feature having a positive correlation with the gait age is selected from the above-described many kinds of features and used for the learning model.

### Score Estimation Unit 24

As illustrated in FIG. 6, the score estimation unit 24 includes a knee condition score estimation unit 51, a gait age score estimation unit 52, and a second display processing unit 53.

### Knee Condition Score Estimation Unit 51

The knee condition score estimation unit 51 estimates, as a part of the health score, a knee condition score for quantitatively evaluating the knee condition of the user as a health status. The knee condition score estimation unit 51 provides the features of the user to the learning model generated by the knee condition learning model generation unit 40, and estimates the knee condition of the user as a knee condition score. Then, the knee condition score estimation unit 51 outputs a signal corresponding to the estimated knee condition score to the second display processing unit 53.

### Gait Age Score Estimation Unit 52

The gait age score estimation unit 52 estimates, as a part of the health score, a gait age score for quantitatively evaluating the gait age of the user as a health status. The gait age score estimation unit 52 provides the features related to the gait condition of the user to the learning model generated by the gait age learning model generation unit 41, and estimates the gait age of the user as a gait age score. Then, the gait age score estimation unit 52 outputs a signal corresponding to the estimated gait age score to the second display processing unit 53.

### Second Display Processing Unit 53

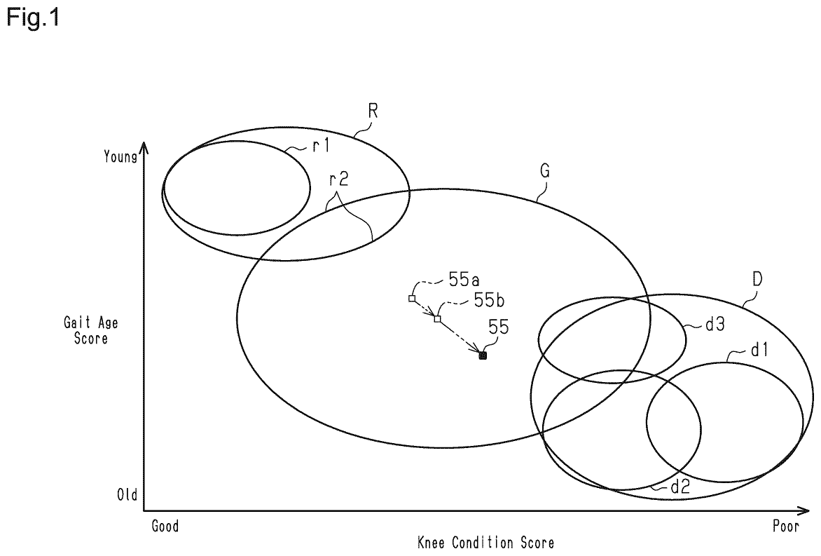

The second display processing unit 53 displays the image of the two-dimensional map shown in FIG. 1 on the display 25.

This map defines the relationship of the health status to the knee condition score and the gait age score. The knee condition score is plotted along the horizontal axis of the map, indicating better knee conditions towards the left side and worse conditions towards the right side. The gait age score is plotted along the vertical axis of the map, showing that higher scores represent a more youthful gait, while lower scores indicate an older gait.

The health status is roughly classified into the following three regions D, G, and R.

The central region G of the map indicates the region in which there are a large number of people with a general status of health. The upper left region R of the map indicates the region in which there are a large number of people with a robust health status. The lower right region D of the map indicates the region in which there are a large number of people who have been diagnosed with illnesses.

The region R includes a region r1 in which there are many students, athletes, and the like, and a region r2 in which there are many young and healthy people. The region R overlaps a part of the region G in the region r2.

The region D includes a region d1 in which there are many people with frailty, a region d2 in which there are many people with knee osteoarthritis, and a region d3 in which there are many people with anterior or posterior cruciate ligament injuries. Frailty is a state characterized by reduced resistance and physical strength due to aging A part of the region d1 and a part of the region d2 overlap with each other. A part of the region d2 and a part of the region d3 overlap with each other.

The region D overlaps with a part of the region G in a part of the region d2, a part of the region d3, or the like.

The regions D, G, and R have been determined by measuring or surveying the health status, knee condition, and gait age of numerous data providers.

The second display processing unit 53 further superimposes a marker 55 such as a dot on the display 25 at a position of two-dimensional coordinates determined by the knee condition score estimated by the knee condition score estimation unit 51 and the gait age score estimated by the gait age score estimation unit 52 on the map of FIG. 1. The superimposed marker 55 indicates the health status of the user.

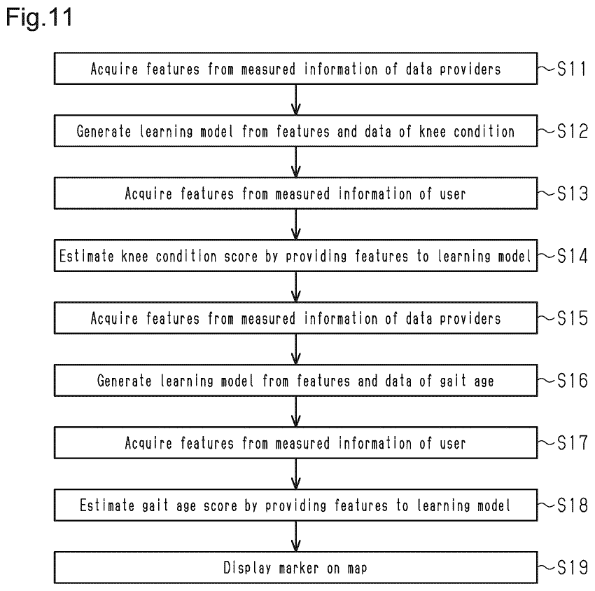

Next, a method of estimating each of the knee condition score and the gait age score of the user as the health score using the gait determination system will be described with reference to the flowchart of FIG. 11.

First, a procedure for estimating the knee condition score will be described.

In a data provider feature acquiring step S11, the gait determination device 20 acquires a feature related to the gait condition of each data provider and having a positive correlation with the knee condition from measurement information indicating temporal changes in pressures applied to multiple parts of the soles of multiple data providers. That is, a feature having a positive correlation with the knee condition is acquired from among the many types of features described above. As described above, the features F1 and F2 are acquired by the interval maximum value acquisition unit 33. The features F3 to F5 are acquired by the interval time acquisition unit 34. The features F6 to F8 are acquired by the dwell ratio calculation unit 36. The features F9 to F11 are acquired by the swing value calculation unit 37. The feature F12 is acquired by the FTA calculation unit 38, and the feature F13 is acquired by the LHA calculation unit 39.

Next, in a learning model generating step S12, the gait determination device 20 generates a learning model by learning the correlation between the features and the knee conditions from the features acquired in the data provider feature acquiring step S11 and the knee conditions of the respective data providers. In other words, rules that describe the relationship between the features and the knee condition are identified, and an algorithm representing these rules is developed as a learning model.

At this time, the results of the data providers' responses to a questionnaire (JKOM, Locomo 25) regarding the knee condition are used as the knee conditions of the data providers. In addition, as the data on the knee condition of each data provider, information related to a classification (Kellgren-Lawrence classification) indicating the degree of progress of a knee disease, for example, knee osteoarthritis is used.

In the subsequent user feature acquiring step S13, the gait determination device 20 acquires features of the same type as the features acquired in the data provider feature acquiring step S11, from measurement information indicating temporal changes in pressures applied to multiple parts of the sole of the user.

Next, in a knee condition score estimation step S14, the gait determination device 20 provides the feature of the user acquired in the user feature acquiring step S13 to the learning model generated in the learning model generating step S12, and estimates an unknown knee condition of the user as the knee condition score.

Next, a procedure for estimating the gait age score will be described.

First, in a data provider feature acquiring step S15, the gait determination device 20 acquires a feature related to the gait condition of each data provider and having a positive correlation with the gait age from measurement information indicating temporal changes in pressures applied to multiple parts of the soles of multiple data providers. That is, a feature having a positive correlation with the gait age is acquired from among many types of the features described above.

Next, in a learning model generating step S16, the gait determination device 20 generates a learning model by learning the correlation between the features and the gait ages from the features acquired in the data provider feature acquiring step S15 and the gait ages of the respective data providers. In other words, rules that describe the relationship between the features and the gait age are identified, and an algorithm representing these rules is developed as a learning model.

In the subsequent user feature acquiring step S17, the gait determination device 20 acquires features of the same type as the features acquired in the data provider feature acquiring step S15, from measurement information indicating temporal changes in pressures applied to multiple parts of the sole of the user.

Next, in a gait age score estimation step S18, the gait determination device 20 provides the feature of the user acquired in the user feature acquiring step S17 to the learning model generated in the learning model generating step S16, and estimates an unknown gait age of the user as the gait age score.

In a display step S19, the gait determination device 20 superimposes the marker 55 such as a dot on the map shown FIG. 1 at a position of two-dimensional coordinates determined by the knee condition score and the gait age score, which have been estimated as described above. The first embodiment has the following advantages.
(1-1) In the present embodiment, the data provider feature is acquired from the measurement information indicating temporal changes in the forces applied to multiple parts of a sole of each of multiple data providers. The data provider feature is related to the gait condition of each data provider and is correlated with the knee condition. The learning model is generated by learning the correlation between the acquired data provider feature and the knee condition from the data provider feature and data on the knee condition of each data provider. The user feature is acquired from the measurement information indicating temporal changes in pressures applied to multiple parts of a sole of the user. The user feature is the same type as the data provider feature, which has been acquired in the above-described manner. The knee condition of the user is estimated as the knee condition score by providing the user feature to the learning model.

Accordingly, it is possible to ascertain the severity of the user's knee condition based on the estimated knee condition score without the need for a direct diagnosis by health professionals. If the knee condition is poor, the user can take appropriate actions such as receiving a detailed diagnosis and treatment at a medical institution. In addition, a person other than the user can take an appropriate action such as prompting the user to have a medical examination at a medical institution. The other person may be, for example, a guardian, a helper, a caregiver, or a health professional.
(1-2) In the present embodiment, the results of the data providers' responses to the questionnaire (JKOM, Locomo 25) regarding the knee condition are used as data on the knee conditions of the data providers. Therefore, the data on the knee conditions of the data providers reflects subjective symptoms actually felt by the data providers with respect to the knee conditions, for example, the subjective symptoms accompanied by pain or the like. The data on the knee conditions thus has high accuracy. Since data with high accuracy is used as described above, the accuracy of the learning model and thus the estimation accuracy of the knee condition score are improved.
(1-3) In the present embodiment, information related to the classification (Kellgren-Lawrence classification) indicating the degree of progression of knee disease is used as the data on the knee conditions of the data providers. Therefore, the data on the knee condition of the data providers objectively reflects the degrees of progress of the disease of the knees, and has high accuracy. Since data with high accuracy is used as described above, the accuracy of the learning model and thus the estimation accuracy of the knee condition score are improved.
(1-4) In the present embodiment, the data provider feature is acquired from the measurement information indicating temporal changes in the forces applied to multiple parts of a sole of each of multiple data providers. The data provider feature is related to the gait condition of each data provider and is correlated with the gait age. The learning model is generated by learning the correlation between the acquired data provider feature and the gait age from the data provider feature and data on the gait age of each data provider. The user feature is acquired from the measurement information indicating temporal changes in pressures applied to multiple parts of a sole of the user. The user feature is the same type as the data provider feature. The gait age of the user is estimated as the gait age score by providing the user feature to the learning model.

Accordingly, it is possible to ascertain the gait age of the user based on the estimated gait age without the need for a direct diagnosis by health professionals. If the estimated gait age significantly deviates towards an older age relative to the actual age, the user can become aware of potential health issues early on, regardless of the presence or absence of subjective symptoms. As in the case of item (1-1) above, if the knee condition is poor, the user can take appropriate actions such as receiving a detailed diagnosis and treatment at a medical institution. In addition, a person other than the user can take an appropriate action such as prompting the user to have a medical examination at a medical institution.
(1-5) In the present embodiment, the two-dimensional map defining the relationship of the health status to the knee condition score and the gait age score is generated and displayed on the display 25.

The estimated knee condition score and the estimated gait age score are displayed on the map with the marker 55 such as a dot. Consequently, by observing the region of the map where the marker 55 is displayed, it is possible to determine which health status region the user falls into.

The user can detect early signs of lumbar diseases, which allows for the possibility to halt the progression of the disease. This enables the user to become aware of necessary improvements much earlier than if the signs were unnoticed.

The above-described first embodiment may be modified as follows. The first embodiment and the following modifications can be combined as long as the combined modifications remain technically consistent with each other.

A health status different from the knee condition and the gait age, for example, a lumbar condition score for quantitatively evaluating the lumbar condition may be estimated as the health score.

The above-described method of estimating the health score is preferably performed multiple times at different times. The execution times may be regular or irregular.

In this case, the knee condition score, the gait age score, and the estimation times thereof are stored in the storage unit 22 at each execution time. Additionally, if there is a history of markers being displayed in the past, these historical markers are also displayed on the map along with the current marker 55. For example, a marker 55a in FIG. 1 indicates a marker that was displayed six months ago, and a marker 55b indicates a marker that was displayed three months ago.

By implementing this method, it becomes possible to understand the temporal changes in health status. For instance, as shown in FIG. 1, if the display positions of the markers shift diagonally downward to the right, from the marker 55a to the marker 55b to the marker 55, it indicates that the health status, particularly the condition of the knee, is deteriorating.

In a case in which the response results to the questionnaire are used as the data of the knee conditions of the data providers, the response results of the data providers to a questionnaire of a type different from the above-described JKOM and Locomo 25 may be used.

In a case in which a classification (stage classification) indicating the degree of progress of a knee disease is used as the data on the knee condition of each data provider, a classification different from the above-described Kellgren-Lawrence classification may be used.

In the flowchart of FIG. 11, only one of the knee condition score and the gait age score may be estimated.

The foot of which the pressure is detected by the pressure sensors 12 may be either one foot or both feet.

The number and arrangement of the pressure sensors 12 provided in the measurement device 10 are not limited to the configuration of the above-described embodiment, and may be appropriately changed according to the type of the features to be acquired. In a case in which the FTA and the LHA are acquired as the features, it is preferable that the pressure sensors 12 be provided at the first position and the second position, which are separated from each other in the longitudinal direction on the sole, and the third position and the fourth position, which are separated from each other in the lateral direction with the line L connecting the first position and the second position, therebetween.

The measurement device 10 may include a storage unit that stores the measurement information. In a case in which a removable recording medium such as a memory card is used as the storage unit of the measurement device 10, the transmission unit 13 of the measurement device 10 and the reception unit 21 of the gait determination device 20 may be omitted.

In the first embodiment and the above modifications, pressure has been described as a type of the force, but the force may be acceleration or torque. The force sensors may be acceleration sensors, torque sensors, or the like capable of measuring these types of force. These sensors may, for example, be incorporated in the base 11.

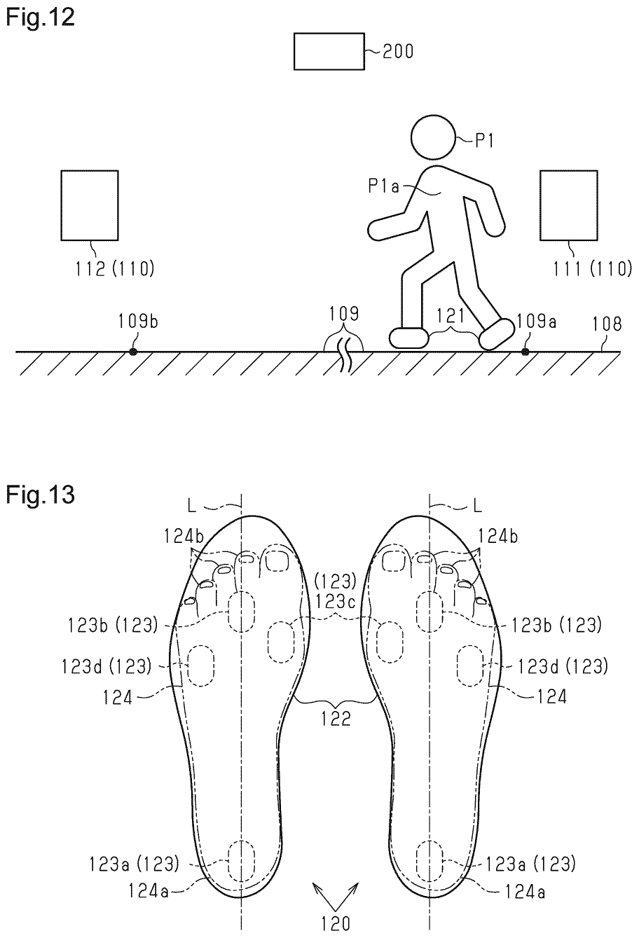

A second embodiment of the present disclosure will now be described with reference to the drawings. The second embodiment relates to a method of estimating changes in positions of parts of upper body. In the estimating method of the present embodiment, a first measurement device 110 shown in FIG. 12 and a second measurement device 120 shown in FIG. 13 are used. Next, each measurement device will be described.

### First Measurement Device 110

As shown in FIG. 12, the first measurement device 110 is installed at a location having a horizontal or nearly horizontal flat surface 108. A portion of the flat surface 108 having a certain width, for example, 10 meters in the lateral direction in FIG. 12 is used as a straight walking path 109 on which a data provider P1 walks. The walking path 109 may be set indoors or outdoors.

The first measurement device 110 includes two image capturing devices 111, 112. An example of each image capturing device 111, 112 is a digital camera, a tablet on which a digital camera is mounted, or the like.

The image capturing devices 111, 112 are installed on both sides in the length direction of the walking path 109 so as to interpose the walking path 109 therebetween. Specifically, the image capturing device 111 is installed near one end 109a of the walking path 109, and the image capturing device 112 is installed near the other end 109b of the walking path 109. The two image capturing devices 111, 112 are installed so as to face each other in the direction in which the walking path 109 extends.

Each of the image capturing devices 111, 112 generates images by capturing images of the data provider P1 walking on the walking path 109. Each image capturing device 111, 112 generates video data consisting of multiple images that are chronologically sequential.

Each image capturing device 111, 112 includes a transmission unit (not shown). The transmission unit of each image capturing device 111, 112 transmits the generated video data to a cloud computing system 200 (hereinafter referred to as a cloud 200) via a network. The cloud 200 may be, for example, processing circuitry such as a computer including a CPU.

Through motion capture of the aforementioned video data, the temporal changes in the positions of multiple parts of the upper body P1a of the data provider P1 are calculated. In the present embodiment, multiple joints of the upper body P1a are used as the multiple parts of the upper body P1a. A joint is a portion at which adjacent bones are connected. Bones can move in various directions with joints as fulcrums. The joints are locations that serve as fulcrums when the bones move, and are locations at which the amounts of changes in positions of the multiple parts are the smallest when the upper body P1a moves. The calculation of the temporal changes in the positions of the multiple parts is performed at any time before the generation of a learning model, which will be discussed below.

### Second Measurement Device 120

As shown in FIGS. 12 and 13, the second measurement device 120 includes insoles 122 each placed on the inner bottom of a shoe 121. The second measurement device 120 can basically have the same configuration as the measurement device 10 shown in FIG. 2. The insole 122 may correspond to the base 11 of the measurement device 10 shown in FIG. 2. Force sensors 123 for detecting forces applied to multiple parts of the sole of a sole of the foot 124 during walking are incorporated in each insole 122. The force sensors 123 are pressure sensors 123 that measure pressures applied to multiple parts of the sole. The pressure sensors 123 include a heel-side sensor 123a, a toe-side sensor 123b, an inner-side sensor 123c, and an outer-side sensor 123d.

As shown in FIG. 13, the heel-side sensor 123a is disposed at a portion of the insole 122 at which the load of a heel 124a is applied, and measures the pressure applied to the heel 124a of the sole. The toe-side sensor 123b is disposed at a position in the insole 122 close to any one of five toes 124b that receives load, and measures pressure applied to the toes 124b of the sole.

The inner-side sensor 123c is disposed on the inner side of an imaginary line L connecting the heel-side sensor 123a and the toe-side sensor 123b in the insole 122, and at a portion to which the load of the ball of the big toe is applied. The inner-side sensor 123c measures the pressure applied to the inner portion of the sole The outer-side sensor 123d is disposed on the outer side of the line L in the insole 122 and at a portion to which the load of the ball of the little toe is applied. The outer-side sensor 123d measures the pressure applied to the outer part of the sole.

The pressure sensors 123 are disposed at the four positions in the insole 122 for the following reason.

In walking, among multiple parts of the sole, the heel 124a touches the ground first, and the part contacting the ground changes in order from the heel 124a toward the toes 124b. Further, after the heel 124a touches the ground and before the toes 124b touch the ground, at least one of a portion on the inner side and a portion on the outer side of the imaginary line L touches the ground. The specific part of the foot that touches the ground affects the resulting toe grip strength.

Thus, in the present embodiment, two of the pressure sensors 123 are respectively incorporated in the portion of the insole 122 to which the load of the heel 124a is applied and the portion to which the load of the toes 124b is applied. Further, the other pressure sensors 123 are respectively incorporated in each of the portions of the insole 122 on the inner side and the outer side of the imaginary line L. Therefore, the pressure sensors 123 incorporated in the insole 122 appropriately measure temporal changes in pressures applied to multiple parts of the sole.

The pressure sensors 123 may be sensors similar to the pressure sensors 12 in the first embodiment.

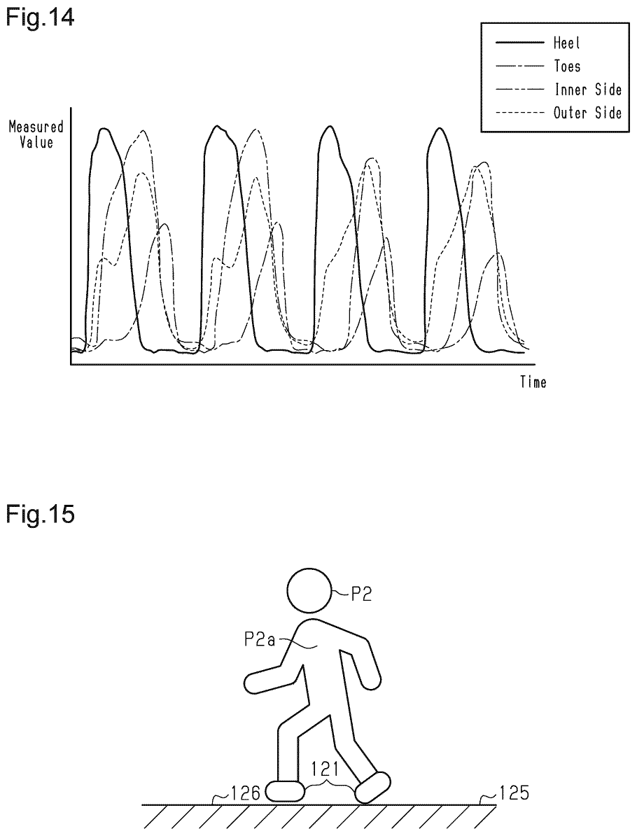

FIG. 14 shows examples of waveforms representing temporal changes in pressures at respective parts of the sole measured by the pressure sensors 123. In FIG. 14, the solid line indicates a measured value of the heel-side sensor 123a, the long-dash short-dash line indicates a measured value of the toe-side sensor 123b, the long-dash double-short-dash line indicates a measured value of the inner-side sensor 123c, and the broken line indicates a measured value of the outer-side sensor 123d.

The insole 122, which incorporates the pressure sensors 123, is used to measure the temporal changes in pressures at multiple parts of the sole. The subjects of this measurement are multiple data providers P1 shown in FIG. 12 and a specified user P2 shown in FIG. 15. In other words, the common insoles 122 are used for the multiple data providers P1 and the user P2, and temporal changes in the pressures applied to the multiple parts of the soles are measured. The common insoles 122 are insoles 122 of the same type, more specifically, insoles 122 in which the same number of pressure sensors 123 are disposed at the same positions.

The measurement of temporal changes in pressures for each data provider P1 shown in FIG. 12 is performed at the same location and at the same time as the measurement of temporal changes in the positions of the multiple parts. Therefore, when a data provider P1 walks on the walking path 109, temporal changes in pressure are measured in addition to temporal changes in the positions of the multiple parts.

The insole 122 includes a transmission unit (not shown) in addition to the pressure sensors 123. The transmission unit transmits pressure data measured by the pressure sensors 123 to the cloud 200 via the network. This transmission is performed at times synchronized with the times at which the video data is transmitted from the transmission units of the image capturing devices 111, 112. For example, the synchronization is performed by using a method specified in a communication protocol such as NTP.

The measurement of temporal changes in pressures on the sole of the user P2 shown in FIG. 15 is conducted while the user P2 walks on a walking path 126 set on a horizontal or nearly horizontal flat surface 125.

The temporal changes in positions related to the data providers P1 measured by the first measurement device 110, and the temporal changes in the pressures related to the data providers P1 measured by the second measurement device 120 are used to estimate the temporal changes in the positions of the multiple parts of the upper body P2a of the user P2.

### Operation of Present Embodiment

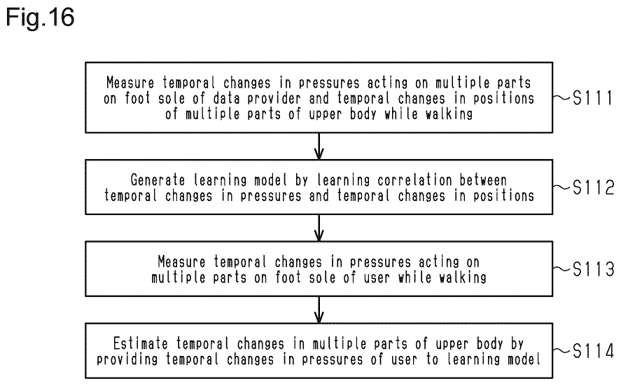

Next, a procedure for estimating temporal changes in the positions of multiple parts of the upper body P2a of the user P2 based on temporal changes in pressures on multiple parts of the sole of the user P2 will be described by referring to the flowchart in FIG. 16.

This estimation employs a technology known as machine learning, which involves training a computer. One of the methods of machine learning is the neural network, which is a mathematical model proposed to mimic the neural circuits of the human brain. In the human brain, numerous nerve cells (neurons) collaborate to exchange electric signals, meaning that the neurons transmit electric signals to each other to perform processes such as thinking and recognition.

Each neuron receives electric signals from a neuron on the input side for electric signals and accumulates the electric signals. Once the accumulated electric charge exceeds a threshold, the neuron transmits an electric signal to a neuron on the output side. The act of transmitting an electric signal to a neuron on the output side when the electric signal exceeds the threshold is referred to as “firing”. Furthermore, each neuron is connected to multiple neurons on both the input and output sides, with the strength of these connections varying depending on the combination of neurons.

A neural network has a structure in which numerous neurons are interconnected. A neural network includes multiple structures called “layers”. These layers include an “input layer”, into which data is entered, an “output layer”, from which results are produced, and “hidden layers” (intermediate layers) located between the input layer and the output layer. Each layer is structured with multiple nodes connected by edges. Nodes in each layer use the outputs from all the nodes in the preceding layer as their inputs.

In a neural network, values are transformed from one layer to the next. A neural network can be considered as a single large function comprised of multiple such transformations. The number of nodes in the input layer and the output layer is determined by the type of data being input and the desired output.

In a neural network, the value of the electric signal received by a specified neuron is adjusted according to the strength of the electric signals entering the neuron and the degree of connectivity between neurons. When the accumulated electric charge in the neuron exceeds a certain threshold, “1” is outputted. This corresponds to the firing of the neuron. That is, the node becomes activated and data is transmitted to the next layer of the network. If the threshold is not exceeded, the data is not transmitted to the next layer.

The aforementioned neural network is applied to “learning” and “inference (estimation)”. Learning involves gradually adjusting the weights of each layer of the neural network to reduce the error with the correct labels, thereby increasing accuracy. Inference (estimation) is the step of using the adjusted network to produce responses. The flowchart in FIG. 16 illustrates these processes of learning and inference (estimation), as does the previously mentioned flowchart in FIG. 11.

In a data provider measurement step S111, as shown in FIG. 12, the data provider P1 puts on shoes 121 that are fitted with the insoles 122 shown in FIG. 13. Before starting to walk, the data provider P1 assumes a stationary posture at one end 109a of the walking path 109 in the longitudinal direction. At this time, namely while the data provider P1 is in the static upright position, each image capturing device 111 and 112 captures images of the data provider P1 for a set duration (for example, approximately 30 seconds) and performs calibration. The calibration here refers to the process of verifying whether the settings and the images captured by the image capturing devices 111 and 112 are correct, using the data of the captured image of the static upright position as a reference, and adjusting settings as necessary to ensure accurate capturing of images.

The data provider P1 walks along the walking path 109, which is set on the horizontal or nearly horizontal flat surface 108, starting from one end 109a toward the other end, 109b. Upon reaching the end 109b, the data provider P1 turns around and walks back toward the end 109a. Then, the data provider P1 moves back and forth on the walking path 109 multiple times, for example, twice, and returns to the end 109a.

During the period when the data provider P1 is walking along the walking path 109, the image capturing devices 111 and 112 capture images, thereby generating video data that includes a series of images in chronological order. The transmission units of the image capturing devices 111, 112 transmit the video data generated by the image capturing devices 111, 112 to the cloud 200 via a network.

While the data provider P1 is walking along the walking path 109, the pressure sensors 123 measure temporal changes in pressures at multiple parts of the sole. Specifically, the heel-side sensor 123a measures the pressure on the heel 124a of the sole, and the toe-side sensor 123b measures the pressure on the toes 124b of the sole. The inner-side sensor 123c measures the pressure on the inner side of the sole, and the outer-side sensor 123d measures the pressure on the outer side of the sole. The transmission unit of each insole 122 then transmits the pressure data measured by the pressure sensors 123 to the cloud 200 via the network.

The aforementioned two types of measurements are conducted on numerous data providers P1. Through these measurements, video data and pressure data of the soles of numerous data providers P1 are acquired.

Next, in a learning model generation step S112 in FIG. 16, the correlation between the temporal changes in the pressures and the temporal changes in the positions of the multiple parts measured in the data provider measurement step S111 is learned. This process involves identifying common characteristics, such as underlying rules, from a large dataset, thereby creating a learning model. These characteristics, numerically expressed as features, are treated as variables that provide clues for estimation in machine learning.

The learning model is an algorithm that represents these rules, essentially a series of procedures to solve problems, and is also referred to as a predictor. During the learning process, the weights representing the connection strengths between neurons are adjusted to minimize errors between output values and correct answers (the values of error functions). For this adjustment, methods such as gradient descent and backpropagation are used to find approximate values of weights that minimize the error function. The weights are updated repeatedly based on the results obtained, progressively bringing the weights closer to the target values.

As the architecture of the neural network, a long short-term memory (LSTM) network acquired by improving a recurrent neural network (RNN) is suitable. An RNN is an extended version of a neural network, specifically designed to handle time-series data. Time-series data refers to a type of data that varies in value over time.

An LSTM enables the retention of the influence of past outputs over a long period of time in an RNN, by introducing the concept of memory span to the outputs of a hidden layer (intermediate layer).

In a user measurement step S113 in FIG. 16, the user P2 wears the shoes 121, which incorporate the same insoles 122 as the insoles 122 used for the pressure measurement of the data providers P1, as shown in FIG. 15. The user P2 walks on the walking path 126, which is set on the horizontal or nearly horizontal flat surface 125. At this time, temporal changes in pressures applied to multiple parts of the sole are measured. This measurement is performed in the same manner as the measurement in the provider measurement step S111. Specifically, during the period when the user P2 is walking, the heel-side sensor 123a measures the pressure applied to the heel 124a, while the toe-side sensor 123b measures the pressure applied to the toes 124b. During the period, the inner-side sensor 123c measures the pressure on the inner side of the sole, and the outer-side sensor 123d measures the pressure on the outer side of the sole. The transmission unit of each insole 122 then transmits the pressure data measured by the pressure sensors 123 to the cloud 200 via the network.

In an estimation step S114 in FIG. 16, the temporal changes in pressure, which were measured in the user measurement step S113, are provided to the learning model generated in the learning model generation step S112 as unknown data. Then, in the estimation step S114, temporal changes in the positions of the multiple parts of the upper body P2a of the user P2 are estimated.

In the multiple procedures previously described in FIG. 16, the learning model generation step S112 and the estimation step S114 are performed by a computer for analysis. The computer for analysis is processing circuitry that may be provided in the cloud 200 or may be provided separately from the cloud 200. The estimation results may be transmitted from the computer for analysis to a computer such as a mobile terminal or a tablet terminal. The estimation results may be displayed on the display of the computer for analysis, or on the display of a computer that has received the estimation results from the computer for analysis. The estimation results may be displayed in the form of a map as condition scores as shown in FIG. 1, for example.

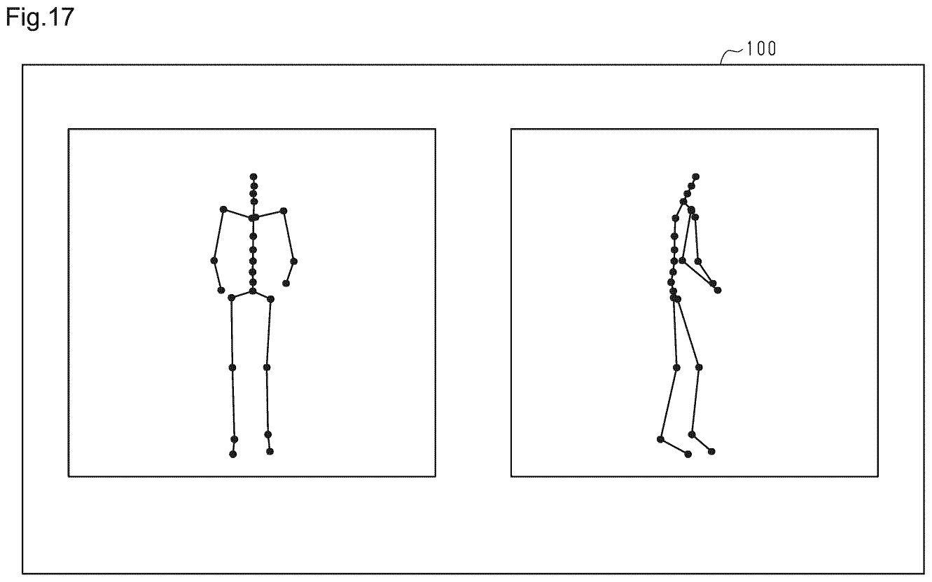

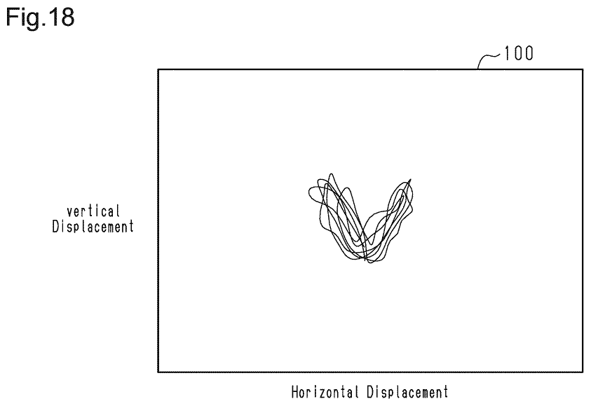

The temporal changes in the positions of one or more parts of the upper body P2a of the user P2 may be displayed on the display based on the estimation results. For example, as shown in FIG. 17, the display 100 may show multiple markers respectively corresponding to the multiple parts of the user P2, and the positions of these markers may change over time on the display 100. The display may show a trajectory of displacement of each of multiple markers respectively corresponding to multiple parts of the user P2. As shown in FIG. 18, as an example, the display 100 may show a trajectory of displacement of a marker corresponding to a specific part in the lumbar region of the user P2.

### Advantages of Second Embodiment

(2-1) Temporal changes in pressures applied to multiple parts of the sole are measured when the data provider P1 walks. In synchronization with this measurement, temporal changes in the positions of the multiple parts of the upper body P1a are measured. A learning model is generated by learning a correlation between the measured temporal changes in the pressures and the measured temporal changes in the positions of the multiple parts. The same insoles 122 used for measuring the pressure for the data provider P1 are also used to measure the temporal changes in pressures on multiple parts of the soles when the user P2 walks. By inputting these measured temporal changes in pressures into the learning model, the temporal changes in the position of the multiple parts of the upper body P2a of user P2 are estimated.

Therefore, when the user P2 walks, temporal changes in the positions of the multiple parts of the upper body P2a can be estimated by measuring temporal changes in the pressures applied to the multiple parts of the soles.
(2-2) From the temporal changes in the positions of the multiple parts of the upper body P2a of the user P2, it is possible to acquire various useful information regarding the physical condition of the user (P2), as detailed below.

Information on the posture of the user P2 is acquired. For instance, information can be obtained about the user P2, such as the fact that the arm is bent, and the torso is leaning forward. From this information, it is possible to recommend actions such as taking appropriate measures before becoming ill, or undergoing a medical check-up for prevention, to the potential subject P2.

Information on the motion of the user P2 is acquired. For instance, it is possible to obtain information such as an estimation that the user P2 is walking while holding a smartphone.

Information can be obtained regarding the discrepancy between the body image (a mental representation of one's own body) present in the brain of the user P2 and the actual condition of the functioning body. The concept of body image includes the function of understanding the contours, size, and position of one's own body. In addition, it encompasses the function of recognizing the extent of one's own physical abilities.

It is believed that there is a correlation between the degree of this discrepancy and the progression of dementia. Therefore, when the discrepancy is identified, it becomes possible to estimate the progression of dementia, for example, mild cognitive impairment (MCI).

The various kinds of information may be displayed in the form of a map as condition scores as shown in FIG. 1, for example. The map may include multiple regions designated to indicate the degree of posture quality for multiple parts of the body of the user subject P2. The state of the estimated posture of each part can be indicated by a marker that is superimposed on the map, showing whether it is located in or near a certain region.
(2-3) In the data provider measurement step S111, the temporal changes in the positions of the multiple parts of the upper body P1a of data provider P1 are measured, focusing specifically on the joints of the upper body P1a. The joints are locations and are locations at which the amounts of changes in the positions of the multiple parts are the smallest when the upper body P1a moves. This allows for measurement of temporal changes in the positions of the multiple parts of the upper body P1a at locations where the movements of the upper body P1a are relatively small.
(2-4) In the process of measuring pressures at multiple points on the soles, the shoes 121 are used that are equipped with the insoles 122 in the inner bottoms. The insoles 122 incorporate the pressure sensors 123. In the data provider measurement step S111, the data provider P1 walks while wearing the shoes 121 equipped with the insoles 122. In the user measurement step S13, the user P2 walks while wearing the shoes 121 equipped with insoles 122. This allows for the accurate measurement of the temporal changes in the pressure applied to multiple parts of the soles.
(2-5) Two of the pressure sensors 123 are respectively incorporated in the portion of the insole 122 to which the load of the heel 124a is applied and the portion to which the load of the toes 124b is applied. Further, the other pressure sensors 123 are respectively incorporated in each of the portions of the insole 122 on the inner side and the outer side of the imaginary line L. Therefore, it is possible to accurately measure the temporal changes in pressures applied to multiple parts of the sole using a small number of pressure sensors 123.

### Modifications

The second embodiment may be modified as follows. The second embodiment and the following modifications can be combined as long as the combined modifications remain technically consistent with each other.

Personal authentication may be performed by using temporal changes in the positions of the multiple parts estimated in the estimation step S114. This authentication may be performed solely based on the estimated temporal changes in the positions of the multiple parts. The authentication may be performed by combining it with other authentication technologies, such as ID cards, fingerprint recognition, and facial recognition. Such a combination of different technologies increases the accuracy of authentication.

For example, when personal authentication is conducted by walking through an authentication gate, pressure sensors that measure the pressure on the soles may be embedded in the floor at or near the authentication gate instead of in the insoles 122. This allows for authentication by simply walking on the floor, without the need for the insoles 122.

Alternatively, a person being authenticated at the authentication gate may be asked to wear the shoes 121 equipped with the insole 122 and walk through the gate.

The feet 124 on which the pressure sensors 123 measure pressures on multiple parts of the soles typically involve both feet 124, although it is also permissible to measure using just one foot 124, either left or right.

The number and arrangement of the pressure sensors 123 incorporated in the insoles 122 may be changed.

In place of the image capturing devices 111, 112, acceleration sensors may be used for the first measurement device 110.

The length of the walking path 109, 126 may be changed. The number of times the data provider P1 walks back and forth on the walking path 109 may also be changed.

In the second embodiment and the above modifications, pressure has been described as a type of the force, but the force may be acceleration or torque. The force sensors may be acceleration sensors, torque sensors, or the like capable of measuring these types of force. These sensors may, for example, be incorporated in the insole 122.

Various changes in form and details may be made to the examples above without departing from the spirit and scope of the claims and their equivalents. The examples are for the sake of description only, and not for purposes of limitation. Descriptions of features in each example are to be considered as being applicable to similar features or aspects in other examples. Suitable results may be achieved if sequences are performed in a different order, and/or if components in a described system, architecture, device, or circuit are combined differently, and/or replaced or supplemented by other components or their equivalents. The scope of the disclosure is not defined by the detailed description, but by the claims and their equivalents. All variations within the scope of the claims and their equivalents are included in the disclosure.

What is claimed is:

1. A method of estimating a health score, the health score being used to quantitatively evaluate a health status related to walking of a user, and the health score including a knee condition score for quantitatively evaluating a knee condition of the user, the estimating method comprising:
acquiring a data provider feature that is a feature related to a gait condition of each of multiple data providers and is a feature correlated with the knee condition from measurement information indicating temporal changes in forces applied to multiple parts of a sole of each of the multiple data providers;
generating a learning model by learning a correlation between the acquired data provider feature and the knee condition from the data provider feature and data on the knee condition of each data provider;
acquiring a user feature that is a feature of the same type as the data provider feature from measurement information indicating temporal changes in forces applied to multiple parts of a sole of the user; and
estimating the knee condition of the user as the knee condition score by providing the learning model with the acquired user feature.

2. The estimating method according to claim 1, wherein the data on the knee condition of the data providers includes information on results of responses made by the data providers to a questionnaire about the knee condition.

3. The estimating method according to claim 1, wherein the data on the knee condition of the data providers includes information regarding a classification indicating a degree of progress of knee disease.

4. The estimating method according to claim 1, further comprising displaying the estimated knee condition as the knee condition score on a display in a form of a map.

5. The estimating method according to claim 4, wherein
multiple regions for indicating a varying degree of the knee condition are set in the map, and
displaying the estimated knee condition on the display includes indicating which region the estimated knee condition is in or which region the estimated knee condition is near by a marker superimposed on the map.

6. The estimating method according to claim 1, wherein the forces are pressures.

7. A method of estimating a health score, the health score being used to quantitatively evaluate a health status related to walking of a user, and the health score including a gait age score for quantitatively evaluating a gait age of the user, the estimating method comprising:
acquiring a data provider feature that is a feature related to a gait condition of each of multiple data providers and is a feature correlated with the gait age from measurement information indicating temporal changes in forces applied to multiple parts of a sole of each of the multiple data providers;
generating a learning model by learning a correlation between the acquired data provider feature and the gait age from the data provider feature and data on the gait age of each data provider;
acquiring a user feature that is a feature of the same type as the data provider feature from measurement information indicating temporal changes in forces to multiple parts of a sole of the user; and
estimating the gait age of the user as the gait age score by providing the learning model with the acquired user feature.

8. The estimating method according to claim 7, further comprising displaying the estimated gait age as the gait age score on a display in a form of a map.

9. The estimating method according to claim 8, wherein
multiple regions for indicating a varying degree of the gait age are set in the map, and
displaying the estimated gait age on the display includes indicating which region the estimated gait age is in or which region the estimated gait age is near by a marker superimposed on the map.

10. The estimating method according to claim 7, wherein the forces are pressures.

11. A method of estimating changes in positions of parts of upper body, comprising:
measuring, when each of multiple data providers walks, temporal changes in forces applied to multiple parts of a sole of the data provider by using a measurement device;
measuring, in synchronization with the measurement of the temporal changes in the forces, temporal changes in positions of multiple parts of the upper body of the data provider;
generating a learning model by learning a correlation between the measured temporal changes in the forces and the measured temporal changes in the positions of the multiple parts;
measuring, when a user walks, temporal changes in forces applied to multiple parts of a sole of the user by using the same measurement device used for measurement for the data provider; and
estimating temporal changes in positions of multiple parts of the upper body of the user by providing the learning model with the temporal changes in the forces measured for the user.

12. The estimating method according to claim 11, wherein the measuring the temporal changes in the positions of the multiple parts in the upper body of the data provider includes measuring temporal changes in positions of multiple joints in the upper body.

13. The estimating method according to claim 11, wherein
the measurement device includes an insole with one or more force sensors incorporated therein,
when each data provider walks with a shoe in which the insole is placed, the temporal changes in the forces applied to the multiple parts of the sole are measured by the one or more force sensors, and
when the user walks with a shoe in which the insole is placed, the temporal changes in the forces applied to the multiple parts of the sole are measured by the one or more force sensors.

14. The estimating method according to claim 13, wherein
the one or more force sensors include:
force sensors respectively incorporated in a portion of the insole to which load of a heel is applied and a portion of the insole to which load of toes is applied; and
force sensors respectively incorporated in portions of the insole located on opposite sides of an imaginary line connecting the heel and the toes, and
the temporal changes in the forces applied to the multiple parts of the sole are respectively measured by the corresponding force sensors.

15. The estimating method according to claim 11, wherein the forces are pressures.

16. The estimating method according to claim 13, wherein the forces are pressures, and the one or more force sensors are one or more pressure sensors.
